(19) United States
(12) Patent Application Publication
(10) Pub. No.: US 2021/0386649 A1
(43) Pub. Date: Dec. 16, 2021

(54) USE OF SPECIFIC POLYMERS TO BRING ABOUT AN ANTI-POLLUTION EFFECT

(71) Applicant: BASF SE, Ludwigshafen (DE)

(72) Inventors: Annette Mehling, Dusseldorf-Holthausen (DE); Heidi Riedel, Dusseldorf-Holthausen (DE); Jan Peter Koch, Dusseldorf-Holthausen (DE); Helga Gondek, Dusseldorf-Holthausen (DE); Peter Hoessel, Ludwigshafen (DE)

(21) Appl. No.: 17/412,368

(22) Filed: Aug. 26, 2021

Related U.S. Application Data
(62) Division of application No. 16/616,270, filed on Nov. 22, 2019.

(30) Foreign Application Priority Data
May 23, 2017 (EP) 17172546.8

(51) Int. Cl. A61K 8/81; A61K 8/06; A61K 8/46; A61Q 5/02; A61Q 19/00

(52) U.S. Cl. CPC A61K 8/8147; A61K 8/06; A61K 8/466; A61K 8/8152; A61K 2800/40; A61K 8/8194; A61Q 5/02; A61Q 19/00; A61K 8/8182

(57) ABSTRACT

The present invention relates to the use of a polymer or of a cosmetic product comprising said polymer, and additionally other known cosmetic ingredients, to bring about an anti-pollution effect on human skin or on human hair, especially to protect human skin or human hair from dust, wherein the polymer is a cationic, anionic or non-ionic polymer, and wherein the polymer has a solubility in water of at least 0.01 g of polymer in 100 g of water at 20° C. at least at a pH in the range between 4 and 9.

### CROSS-REFERENCE TO RELATED APPLICATIONS

This application is a divisional of U.S. patent application Ser. No. 16/616,270 filed Nov. 22, 2019, which is the U.S. National Stage application of International Patent Application No. PCT/EP2018/059581, filed Apr. 13, 2018, which claims the benefit of European Patent Application No. 17172546.8, filed May 23, 2017.

The present invention relates to the use of a polymer or of a cosmetic product comprising said polymer, and additionally other known cosmetic ingredients, to bring about an anti-pollution effect on human skin or on human hair, especially to protect human skin or human hair from dust, wherein the polymer is a cationic, anionic or non-ionic polymer, and wherein the polymer has a solubility in water of at least 0.01 g of polymer in 100 g of water at 20° C. at least at a pH in the range between 4 and 9.

The air surrounding humans is frequently affected by air pollution. One constituent part of air pollution are particulate substances, among these dust and sand, among these also fine dusts, among these fine particulates.

From a cosmetic point of view, the protection of human skin or human hair from air pollution, especially from deposition of dust from the air, is desirable since, inter alia, the appearance of the skin or hair can be impaired by deposition of dust, for example the skin can appear gray or grayish. Adhesion of particles and substances on the particles can also lead to other effects, such as to premature skin aging (e.g. age spots), to skin surface changes such as dry and/or rough skin, to uneven skin tone, to reduced radiance, to an increase of transepidermal water loss, and also to reduced skin elasticity, skin softness and skin suppleness. Changes to hair can also be engendered such as an increase in brittleness and roughness. The sensitivity of the skin and/or the scalp can also be increased.

There is therefore a need for cosmetic products which protect the skin or hair from air pollution, especially from particulate substances, especially sand or dust, especially from fine dusts. Such protection is referred to in the cosmetics industry as an anti-pollution effect on human skin or on human hair. In other words, therefore, a need exists for cosmetic products which achieve an anti-pollution effect on human skin or on human hair.

U.S. Pat. No. 4,913,897 discloses a hydrophilic hydrogel as a protective film against pollution and against toxic substances. Disclosed are hydrogels composed of polyurethanes, of polyacrylonitrile and of copolymers of acrylonitrile and of polyvinyl acetate. Pollution is understood to mean toxic substances and infectious substances.

DE 31 47 024 A1 discloses a film-forming matrix (disclosed are polyethylene oxide, polysaccharides, polyvinyl alcohol) with inorganic particles (talc, mica, chalk) having a diameter of 10 to 200 micrometers for protection from pollution. Pollution is understood to mean poisonous gases and liquids.

EP 1 684 710 A1 discloses an oil gel with a polymer (a water-repelling substance with a water-absorbing powder).

EP 0 884 047 A1 discloses the use of polyamino polymers for protection from light-induced peroxidation of lipids and proteins.

EP 1 157 683 A1 discloses fibers (polyester, polyurethane, acrylic, cotton and others) in cosmetic preparations for protection from air pollution, ozone, carbon monoxide, nitrogen oxides and heavy metals. The fibers disclosed are not soluble in water.

EP 0 577 718 A1 discloses sphingolipids (ceramides and others) in cosmetic preparations for protection from pollution (air pollution, ozone, carbon monoxide, nitrogen oxides and heavy metals).

EP 1 447 074 A1 discloses the use of crosslinked polymeric nanoparticles having a diameter of 1-10 nm in cosmetic products (e.g. skin care products).

KR 101 653 755 discloses the use of a silicone-acrylate copolymer for anti-pollution purposes. FR 2 836 633 discloses the use of a composition comprising a poly-N-vinylpyrrolidone homopolymer for anti-pollution purposes.

The present invention has the object of providing suitable substances for cosmetic application which achieve an anti-pollution effect on human skin or on human hair, that is to say to provide suitable substances for cosmetic application which protect the skin or hair from air pollution, especially from dust, especially from fine dust, preferably in that these substances reduce the adhesion of those substances which constitute air pollution, especially dust, especially fine dust, to the skin or hair.

This object is achieved by the use according to patent claim 1. The use according to patent claim 1 is a subject matter of the present invention. The dependent claims are preferred embodiments of the present invention. The glass transition temperature Tg can be determined as described in the “examples” section.

A number of the polymers according to the invention have the further advantage that they improve the capacity of those substances which constitute air pollution, especially dust and also sand, especially fine dust, to be washed out with water.

Further embodiments of the present invention are given by the uses according to the patent claims, wherein in each case the use for bringing about an anti-pollution effect is specified for the use for reducing the adhesion of those substances which constitute air pollution, especially dust, especially fine dust, on the skin.

Further embodiments of the present invention are given by the uses according to the patent claims, wherein in each case the use for bringing about an anti-pollution effect is specified for the use for improving the capacity of those substances which constitute air pollution, especially dust, especially fine dust, to be washed out with water.

A further subject matter of the present invention and further preferred embodiments of this subject matter are a method for bringing about an anti-pollution effect on human skin or on human hair, especially to protect human skin or human hair from dust, especially from fine dust, comprising the application of the polymer as defined in any of the patent claims—or the application of a cosmetic product comprising the polymer as defined in any of the patent claims and additionally comprising other known cosmetic ingredients - on such human skin or such human hair which require an anti-pollution effect, especially protection from dust, especially protection from fine dust.

The other known cosmetic ingredients which are present in the cosmetic product specified, in addition to the polymer specified, can be water, surfactants, emulsifiers, inorganic or organic fillers, sensory feel additives (substances, the addition of which are intended to modulate the sensory properties, e.g. silicones, powder raw materials, elastomers, actives (e.g. menthol), etc.), pigments, light protection filters, moisturizing substances (e.g. polyols, especially glycerol, urea), water-soluble or fat-soluble active ingredients, additives (e.g. preservatives, fragrances, chelating agents or neutralizing agents), consistency regulators (e.g. fatty alcohols, glyceryl fatty acid esters).

The cosmetic product specified comprising the polymer specified and other known cosmetic ingredients can be in particular an aqueous preparation, for example an aqueous solution or an aqueous gel (referred to as a hydrogel), wherein this preparation preferably comprises 0 to at maximum 20% by weight, preferably 0—at maximum 10%, preferably 0-5% by weight, particularly preferably 0—at maximum 2.5% by weight hydrophobic constituents. Hydrophobic constituents are understood to mean oils, fats and waxes.

The specified cosmetic product comprising the specified polymer and other known cosmetic ingredients may be in particular a semi-solid gel, an aqueous gel, a pump spray, an aerosol spray, an emulsion, such as a cream or a lotion for example.

The specified cosmetic product comprising the specified polymer and other known cosmetic ingredients can be one that can be applied via brush/sponge/metal/plastic applicators, cloths (e.g. wipes), spray applications or by hand.

The specified cosmetic product comprising the specified polymer and other known cosmetic ingredients can be one which can be used as a skin care product for protecting skin from particulate substances and for skin care.

The specified cosmetic product comprising the specified polymer and other known cosmetic ingredients can be one which can be used as a “top coat” additive which is applied after application of a skin care product (such as day creams, sunscreen creams, make-up, foundation etc.), and thereby reduces in particular the adhesion of particulate substances (dust) and optionally improves their ability to be removed.

The specified cosmetic product comprising the specified polymer and other known cosmetic ingredients can be one which can be used as a “base coat” which is applied prior to application of a skin care product (such as day creams, sunscreen creams, make-up, foundation etc.), and thereby reduces in particular the adhesion of particulate substances (dust) and optionally improves their ability to be removed.

The other known cosmetic ingredients which may be used in the specified cosmetic product in addition to the specified polymer (the product is also referred to below as a preparation), can be selected from one or more of the ingredients described below.

The cosmetic preparations can be formulations for body care, for example a body milk, creams, lotions, sprayable emulsions, products for eliminating body odor, etc. The polymers according to the invention can also be used in surfactant-containing formulations, such as, for example, foam baths, shower gels, shampoos and conditioners. According to the end application, the cosmetic preparations comprise a series of further assistants and additives, for example surfactants, further oil bodies, emulsifiers, pearlescent waxes, consistency regulators, thickeners, superfatting agents, stabilizers, polymers, fats, waxes, lecithins, phospholipids, biogenic active ingredients, antidandruff agents, film formers, swelling agents, insect repellents, self-tanning agents, tyrosinase inhibitors (depigmenting agents), hydrotropes, solubilizers, preservatives, perfume oils, dyes, etc., which are listed below by way of example.

All of the details now following up to the start of the “examples” section only describe particular embodiments of the present invention and are not in any case mandatory features of the present invention.

### Polyols and Skin Moisturizing Active Ingredients

These can be, in particular, alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, further alcohols of low carbon number, e.g. ethanol, isopropanol, 1,2-propanediol, glycerol. In addition to the polyols, further skin moisturizing active ingredients such as urea, hyaluronic acid, glyceryl glucoside, serine and others can be used.

### Active Ingredients

These can be understood to mean, in particular, tocopherol, tocopherol acetate, tocopherol palmitate, ascorbic acid, (deoxy)ribonucleic acid and the fragmentation products thereof, β-glucans, retinol, bisabolol, allantoin, phytantriol, panthenol, AHA acids, amino acids, ceramides, pseudoceramides, essential oils, plant extracts, for example aloe vera, prunus extract, bambara nut extract and vitamin complexes. Examples of useful insect repellents include N,N-diethyl-m-toluamide, 1,2-pentanediol or ethyl 3-(N-n-butyl-N-acetylamino)propionate, which is sold under the Insect Repellent® 3535 name by Merck KGaA, and butylacetylaminopropionates.

Possible tyrosine inhibitors, which prevent the formation of melanin and are applied in depigmenting compositions, are, for example, arbutin, ferulic acid, kojic acid, coumaric acid and ascorbic acid (vitamin C).

Particularly preferred active ingredients are plant extracts such as Moringa pteryosperma seed extract (for example Purisoft™ LS 9726 from BASF), Argania spinosa leaf extract (for example Arganyl™ LS 9781 and ARGANYL PW LS 9830 from BASF), Eperua falcata bark extract (for example Eperuline™ PW from BASF).

### Antiperspirants

Antiperspirants are salts of aluminum, of zirconium or of zinc. Such suitable antihydrotic active ingredients are, e.g. aluminum chloride, aluminum chlorohydrate, aluminum dichlorohydrate, aluminum sesquichlorohydrate and complexes thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complexes thereof, for example with amino acids such as glycine. Preference is given to using aluminum chlorohydrate, aluminum zirconium tetrachlorohydrate, aluminum zirconium pentachlorohydrate and complexes thereof. The preparations according to the invention may comprise the antiperspirants in amounts of 1 to 50%, preferably 5 to 30% and especially 8 to 25% by weight—based on the total weight of the cosmetic preparation.

### Esterase Inhibitors

In the presence of perspiration in the underarm region, bacteria form extracellular enzymes—esterases, preferably proteases and/or lipases—which cleave esters present in the perspiration and thus release odorants. Suitable esterase inhibitors are preferably trialkyl citrates such as trimethyl citrate, tripropyl citrate, triisopropyl citrate, tributyl citrate and especially triethyl citrate (Hydagen® CAT, BASF Personal Care and Nutrition GmbH, Dusseldorf/FRG). The substances inhibit enzyme activity and hence reduce odor formation. Further substances which are possible esterase inhibitors are sterol sulfates or phosphates, for example sulfates or phosphates of lanosterol, of cholesterol, of campesterol, of stigmasterol and of sitosterol, dicarboxylic acids and esters thereof, for example glutaric acid, monoethyl glutarate, diethyl glutarate, adipic acid, monoethyl adipate, diethyl adipate, malonic acid and diethyl malonate, hydroxycarboxylic acids and esters thereof, for example citric acid, malic acid, tartaric acid or diethyl tartrate, and zinc glycinate.

The preparations according to the invention may comprise the esterase inhibitors in amounts of 0.01 to 20%, preferably 0.1 to 10% and especially 0.3 to 5% by weight—based on the total weight of the cosmetic preparation.

Deodorizing active ingredients counteract body odors, conceal or remove them. Body odors arise through the action of skin bacteria on apocrine perspiration, which forms unpleasant-smelling degradation products. Correspondingly suitable as deodorizing active ingredients are, inter alia, germination inhibitors, enzyme inhibitors, odor absorbers or odor masking agents.

### Bactericidal or Bacteriostatic Active Ingredients

Typical examples of suitable bactericidal or bacteriostatic active ingredients are especially chitosan and phenoxyethanol. 5-Chloro-2-(2,4-dichlorophenoxy)phenol has also been found to be particularly effective, and is sold under the Irgasan® brand by Ciba-Geigy, Basle, Switzerland. Suitable germicides are in principle all substances which act against Gram-positive bacteria, for example 4-hydroxybenzoic acid and the salts and esters thereof, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynylbutyl carbamate, chlorhexidine, 3,4,4′-trichlorocarbanilide (TTC), antibacterial odorants, thymol, thyme oil, eugenol, clove oil, menthol, mint oil, farnesol, phenoxyethanol, glyceryl monocaprate, glyceryl monocaprylate, glyceryl monolaurate (GML), diglyceryl monocaprate (DMC), N-alkylsalicylamides, for example n-octylsalicylamide or n-decylsalicylamide.

The preparations according to the invention may comprise the bactericidal or bacteriostatic active ingredients in amounts of 0.01 to 5% and preferably 0.1 to 2% by weight—based on the total weight of the cosmetic preparation.

### Perspiration-Absorbing Substances

Perspiration-absorbing substances include modified starch, for example Dry Flo Plus (from National Starch), silicates, talc and other substances of similar modification which appear suitable for absorption of perspiration. The preparations according to the invention may comprise the perspiration-absorbing substances in amounts of 0.1 to 30%, preferably 1 to 20% and especially 2 to 8% by weight—based on the total weight of the cosmetic preparation.

Use may be made, as superfatting agents, of substances such as, for example, lanolin and lecithin, and also polyethoxylated or acylated lanolin and lecithin derivatives, polyol fatty acid esters, monoglycerides and fatty acid alkanolamides in addition to vaseline and paraffins and paraffin waxes, the fatty acid alkanolamides simultaneously serving as foam stabilizers.

### UV Filters

Suitable UV light protection filters in accordance with the invention are organic substances (light protection filters), liquid or crystalline at room temperature, which are capable of absorbing ultraviolet rays and releasing the energy absorbed again in the form of longer-wave radiation, for example heat. UV filters may be oil-soluble or water-soluble. Examples of typical oil-soluble UV-B filters or broad-spectrum UV-A/B filters include:
3-benzylidenecamphor or 3-benzylidenenorcamphor (Mexoryl SDS 20) and derivatives thereof, e.g. 3-(4-methylbenzylidene)camphor, as described in EP 0693471 B1
3-(4′-trimethylammonium)benzylidenebornan-2-one methylsulfate (Mexoryl SO)
3,3′-(1,4-phenylenedimethine)bis(7,7-dimethyl-2-oxobicyclo[2.2.1]heptane-1-methanesulfonic acid) and salts (Mexoryl SX)
3-(4′-sulfo)benzylidenebornan-2-one and salts (Mexoryl SL)
polymer of N-{(2 and 4)-[2-oxoborn-3-ylidene)methyl}benzyl]acrylamide (Mexoryl SW) 2-(2H-benzotriazol-2-yl)-4-methyl-6-(2-methyl-3-(1,3,3,3-tetramethyl-1-(trimethylsilyloxy)disiloxanyl)propyl)phenol (Mexoryl SL)
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzalmalonate;
triazine derivatives, for example 2,4,6-trianilino(p-carbo-2′-ethyl-1′-hexyloxy)-1,3,5-triazine and 2,4,6-tris[p-(2-ethylhexyloxycarbonyl)anilino]-1,3,5-triazine (Uvinul T 150), as described in EP 0818450 A1, or bis(2-ethylhexyl) 4,4′-[(6-[4-((1,1-dimethylethyl)aminocarbonyl)phenylamino]-1,3,5-triazine-2,4-diyl)diimino]benzoate (Uvasorb® HEB);
2,2-(methylenebis(6-(2H-benzotriazol-2-yl)-4-(1,1,3,3-tetramethylbutyl)phenol) (Tinosorb M);
2,4-bis[4-(2-ethylhexyloxy)-2-hydroxyphenyl]-6-(4-methoxyphenyl)-1,3,5-triazine (Tinosorb S);
propane-1,3-diones, for example 1-(4-tert-butylphenyl)-3-(4′-methoxyphenyl)propane-1,3-dione;
ketotricyclo(5.2.1.0)decane derivatives, as described in EP 0694521 B1;
dimethicodiethyl benzalmalonates (Parsol SLX).
tris-biphenyltriazine (TinosorbA2B).

Useful water-soluble UV filters include:
2-phenylbenzimidazole-5-sulfonic acid and the alkali metal, alkaline earth metal, ammonium, alkylammonium, alkanolammonium and glucammonium salts thereof;
2,2-(1,4-phenylene)bis(1H-benzimidazole-4,6-disulfonic acid, monosodium salt) (Neo Heliopan AP);
sulfonic acid derivatives of benzophenones, preferably 2-hydroxy-4-methoxybenzophenone-5-sulfonic acid and salts thereof;
sulfonic acid derivatives of 3-benzylidenecamphor, for example 4-(2-oxo-3-bornylidenemethyl)benzenesulfonic acid and 2-methyl-5-(2-oxo-3-bornylidene)sulfonic acid and salts thereof.

Useful typical UVA filters are especially derivatives of benzoylmethane, for example 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF), and also benzoic acid 2-[4-(diethylamino)-2-hydroxy-benzoyl]hexyl ester (Uvinul® A plus).

The UVA and UVB filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Combinations of this type are advantageously combined with water-soluble filters, for example 2-phenylbenzimidazole-5-sulfonic acid and the alkali metal, alkaline earth metal, ammonium, alkylammonium, alkanolammonium and glucammonium salts thereof.

Suitable UV light protection filters are especially the substances approved according to Annex VII of the Commission Directive (in the version of the EU guideline 2005/9/EC of Jan. 28, 2005 amending guideline 76/768/EEC, concerning cosmetic products, for the purposes of adapting Annex VII to technical progress), to which reference is explicitly made here.

In addition to the soluble substances mentioned, insoluble light protection pigments, specifically finely dispersed metal oxides and salts, are also useful for this purpose. Examples of suitable metal oxides are especially zinc oxide and titanium dioxide, and additionally oxides of iron, of zirconium, of silicon, of manganese, of aluminum and of cerium, and mixtures thereof. The salts used may be silicates (talc), barium sulfate or zinc stearate. The oxides and salts are used in the form of the pigments for skincare and skin-protecting emulsions, and also for decorative cosmetics. The particles should have a mean diameter of less than 100 nm, preferably between 5 and 50 nm and especially between 15 and 30 nm. They may have a spherical shape, but it is also possible to use those particles which have an ellipsoidal shape or a shape which deviates in some other way from the spherical configuration. The pigments may also be present in surface-treated form, i.e. hydrophilized or hydrophobized. Typical examples are coated titanium dioxides, for example T 805 titanium dioxide (Degussa) or Eusolex® T, Eusolex® T-2000, Eusolex® T-Aqua, Eusolex® AVO, Eusolex® T-ECO, Eusolex® T-OLEO and Eusolex® T-S (Merck). Typical examples are zinc oxides, for example Zinc Oxide neutral, Zinc Oxide NDM (Symrise) or Z-Cote® (BASF) or SUNZnO-AS and SUNZnO-NAS (Sunjun Chemical Co. Ltd.). Suitable hydrophobic coating agents are in particular silicones and specifically trialkoxyoctylsilanes or simethicones. In sunscreen compositions, preference is given to using so-called micropigments or nanopigments. Preference is given to using micronized zinc oxide. Further suitable UV light protection filters are given in the overview by P. Finkel in SÖFW-Journal 122, 8/1996, pp. 543-548 and Parf.Kosm 80. No. 3/1999, pp. 10 to 16.

In addition to the two aforementioned groups of primary light protection substances, it is also possible to use secondary light protection agents of the antioxidant type, which interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. -carotene, -carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to mol/kg), also (metal) chelating agents (e.g. α-hydroxy fatty acids, palmitic acid, phytic acid, lactoferrin), α-hydroxy acids (e.g. citric acid, lactic acid, malic acid), humic acid, gallic acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids), suitable in accordance with the invention, of these specified active ingredients.

A preferred embodiment of the invention relates to cosmetic preparations comprising one or more polymers according to the invention and at least one UV light protection filter selected from the group consisting of 4-Methybenzylidene Camphor, Benzophenone-3, Butyl Methoxydibenzoylmethane, Bis-Ethylhexyloxyphenol Methoxyphenyl Triazine, Methylene Bis-Benzotriazolyl Tetramethylbutylphenol, Tris-Biphenyl Triazine, Diethylhexyl Butamido Triazone, Ethylhexyl Triazone and Diethylamino Hydroxybenzoyl Hexyl Benzoate, 3-(4′-trimethylammonium)benzylidenebornan-2-one methylsulfate, 3,3′-(1,4-phenylenedimethine)bis(7,7-dimethyl-2-oxobicyclo-[2.2.1]heptane-1-methanesulfonic acid) and salts thereof, 3-(4′-sulfo)benzylidenebornan-2-one and salts thereof, polymer of N-{(2 and 4)-[2-oxoborn-3-yliden)methyl}benzyl]acrylamide, 2-(2H-benzotriazol-2-yl)-4-methyl-6-(2-methyl-3-(1,3,3,3-tetramethyl-1- (trimethylsilyloxy)disiloxanyl)propyl)phenol, dimethicodiethyl benzalmalonate, insoluble light protection pigments and mixtures thereof.

These UV light protection filters are commercially available, for example, under the following trade names:

NeoHeliopan® MBC (INCI: 4-Methylbenzylidene Camphor; manufacturer: Symrise); NeoHeliopan® BB (INCI: Benzophenone-3, manufacturer: Symrise); Parsol® 1789 (INCI: Butyl Methoxydibenzoylmethane, manufacturer: Hoffmann-La Roche (Givaudan); Tinosorb® S (INCI: Bis-Ethylhexyloxyphenol Methoxyphenyl Triazine); Tinosorb® M (INCI: Methylene Bis-Benzotriazolyl Tetramethylbutylphenol): manufacturer: BASF; Uvasorb® HEB (INCI: Diethylhexyl Butamido Triazone, manufacturer: 3V Inc.), Uvinul® T 150 (INCI: Ethylhexyl Triazone, manufacturer: BASF AG); Uvinul® A plus (INCI: Diethylamino Hydroxybenzoyl Hexyl Benzoate: manufacturer: BASF AG; Mexoryl® SO: 3-(4′-trimethylammonium)benzylidenebornan-2-one methylsulfate INCI: Camphor Benzalkonium Methosulfate; Mexoryl®SX: 3,3′-(1,4-phenylenedimethine)bis(7,7-dimethyl-2-oxobicyclo[2.2.1]heptane-1-methanesulfonic acid), CTFA: INCI Terephthalylidene Dicamphor Sulfonic Acid; Mexory® SL: 3-(4′-sulfo)benzylidenebornan-2-one, INCI Benzylidene Camphor Sulfonic Acid; Mexoryl® SW: polymers of N-{(2 and 4)-[2-oxoborn-3-ylidene)methyl}benzyl]acrylamid, INCI Polyacrylamidomethyl Benzylidene Camphor; Mexoryl® SL: 2-(2H-benzotriazol-2-yl)-4-methyl-6-(2-methyl-3-(1,3,3,3-tetramethyl-1-(trimethylsilyloxy)disiloxanyl)propyl)phenol; INCI: DROMETRIZOLE TRISILOXANE; Parsol® SLX: Dimethicodiethylbenzalmalonate, INCI Polysilicone-15.

Further examples are pigmentary light filters, such as inorganic pigmentary light filters, such as titanium dioxide and zinc oxide and/or organic pigmentary light filters such as methylenebisbenzotriazolyltetramethylbutylphenol (Tinosorb M) and Tris-Biphenyl Triazine (Tinosorb A2B).

The preparations according to the invention may comprise the UV light protection filters in amounts of 0.5 to 30% by weight, preferably 2.5 to 20% by weight, particularly preferably 5-15% by weight—based on the total weight of the cosmetic preparation.

### Self-Tanning Agents

Self-tanning agents are understood to mean substances which cause browning of the skin. Examples include dihydroxyacetone, erythrulose and alpha, beta-unsaturated aldehydes, which react with the amino acids in the skin in the manner of a Maillard reaction to give colored compounds. Useful further active ingredients for self-tanning agents include natural or synthetic ketols or aldols. Examples of suitable active ingredients include dihydroxyacetone, erythrulose, glycerolaldehyde, alloxane, hydroxymethylglyoxal, gamma-dialdehyde, 6-aldo-D-fructose, ninhydrin and meso-tartaraldehyde. Suitable self-tanning agents are especially dihydroxyacetone and/or erythrulose.

Particularly advantageous self-tanning agents have been found to be mixtures of the abovementioned active ingredients with one another or with muconaldehyde or/and naphthoquinones, for example 5-hydroxy-1,4-naphthoquinone (juglone) and 2-hydroxy-1,4-naphthoquinone.

The self-tanning agents are typically present at concentrations of 1 to 10%, especially 2 to 5% by weight—based on the total weight of the cosmetic preparation.

### Pigments

The term pigment encompasses particles of any kind which are white or colored, organic or inorganic, are insoluble in the preparations, and serve the purpose of coloring the preparation. In a preferred embodiment, inorganic pigments are used, particular preference being given to metal oxides.

Examples of inorganic pigments include: titanium dioxide, optionally surface-coated, zirconium or cerium oxides, and zinc, iron (black, yellow or red) and chromium oxides, manganese violet, ultramarine blue, chrome hydrates and iron(III) blue, metal powders such as aluminum powder or copper powder.

In a preferred embodiment of the invention, the pigment is selected from the inorganic pigments, preferably from the metal oxides. In a preferred embodiment, the pigment is selected from the group consisting of titanium dioxide, zinc oxide, iron oxide and mixtures thereof. The inorganic pigments can be colorants. Particularly preferred inorganic coloring pigments are metal oxides and especially iron oxide.

The pigments may be present either individually or in mixtures.

Preference is given in the context of the present invention to pigment mixtures composed of white pigments (e.g. kaolin, titanium dioxide or zinc oxide) and inorganic color pigments (e.g.

iron oxide pigments, chromium oxides), and the pigments may be present in coated or uncoated form. Among the color pigments, iron oxides are particularly preferred.

Advantageously in the context of the present invention, the pigment(s) may also be selected from the group of the effect pigments which impart to the cosmetic preparation, in addition to the pure color, an additional property—for example angular dependence of the color (shimmer, flop), luster (not surface gloss) or texture. Such effect pigments are used in accordance with the invention advantageously in addition to one or more white and/or color pigments.

The most important group of the effect pigments is that of the luster pigments, which, according to DIN 55944: 2003-11 include the metal effect pigments and the pearlescent pigments. Some specific effect pigments cannot be assigned to these two groups, for example graphite platelets, iron oxide platelets and micronized titanium dioxide, the latter not giving a luster effect, but rather an angle-dependent light-scattering effect. In the luster pigments in accordance with DIN 55943: 2001-10, they predominantly take the form of effect pigment platelets. Aligned in parallel, luster pigments exhibit a characteristic luster. The visual effect of luster pigments is based on the directed reflection on metallic particles (metal effect pigments), on transparent particles with a high refractive index (pearlescent pigments) or on the phenomenon of interference (interference pigments) (DIN 55944: 2003-11).

Examples of commercial effect pigments preferred in accordance with the invention are: Timiron and #174; from Merck, Iriodin and #174; from Merck (pearlescent and color luster pigments for decorative industrial applications), Xirallic and #174; from Merck (intense-color crystal effect pigments).

The following pigments can also be used in accordance with the invention:

pigments based on mica:

Cellini®, Cloisonné®, Duocrome®, Flamenco®, Gemtone®, Timica®, MultiReflections™

The substrate mica is with metal oxides—titanium dioxide (TiO₂) and/or iron oxide (Fe₂O₃), in order to generate white interfering and metallic effects. The reflecting color depends on the thickness of the metal oxide coating. To generate absorption and complex color shift effects, additional pigments are also used. These inorganic and organic pigments are chromium oxide, iron oxides, iron ferrocyanide (iron blue), Carmine and D&C/FD&C paints.

Pigments based on calcium sodium borosilicate: platelets of Reflecks™, Reflecks™ Dimensions, and Reflecks™ MultiDimensions or other dyes are coated with metal oxides and/or other colorants in order to generate effect pigments with enhanced color purity, brightness, transparency and reflectivity. Reflecks·8 MultiDimensions consist of platelets composed of calcium sodium borosilicate, which are coated with titanium dioxide and silicon dioxide (SiO₂). These pigments enable distinctive color shift effects.

Pigments based on synthetic mica:

Chione™

Metal oxides are applied to substrates composed of synthetic mica. Owing to the clean main color of the substrate, the appearance formed therefrom is characterized by very bright colors with enhanced chroma. These pigments based on synthetic mica are ideal for use in such products where broad pearlescent effects and iridescent effects are desired.

Pigments based on bismuth oxychloride:

Biju®, Mearlite®, Pearl-Glo®, Chroma-Lite®

Platelets of bismuth oxychloride crystals are precipitated from a solution. Each grade has its own size, shape, thickness and shimmer. Also possible are pigments from the Chroma-Lite range, where bismuth oxychloride are combined with mica and additional colorants.

Specialty Performance Minerals:

Mearlmica®, Chione™ M-SVA, Bi-Lite®,

Specialty performance minerals are a family of mineral products which yield a balance and consistency in certain types of cosmetic compositions, while these remain optically neutral. They also provide improved texture, especially in powder applications. In addition, the preparations according to the invention may advantageously also comprise organic color pigments, i.e. organic dyes which are virtually insoluble in the preparation. In accordance with DIN 55944: 1990-04, organic pigments can be divided according to chemical aspects into azo pigments and polycyclic pigments, and according to color aspects into chromatic or black pigments. Organic white pigments are of no practical significance.

The pigments can advantageously also be used in the context of the present invention in the form of commercially available oily or aqueous predispersions.

The inventive preparations comprise typically 0.1 to 40% by weight of pigments—based on the total weight of the cosmetic preparation.

It is also advantageous in the context of the present invention when the inventive preparation comprises one or more dyes.

The dyes may be either of synthetic or natural origin. A list of suitable dyes can be found in EP 1 371 359 A2, page 8 lines 25-57, page 9 and page 10, and also page 11 lines 1 to 54, to which reference is hereby explicitly made.

The preparations according to the invention comprise typically 0.01 to 5%, preferably 0.1 to 1.0% by weight dyes—based on the total weight of the cosmetic preparation. The preparations according to the invention typically comprise a total amount of dyes and pigments in the range from 0.01 to 30% by weight, especially 0.1 to 15% by weight, preferably 1 to 10% by weight, based on the total weight of the cosmetic preparation.

Suitable dyes and pigments are especially the dyes and pigments approved according to Annex IV of the Commission Directive (in the version: EU guideline 2005/9/EC of Jan. 28, 2005 amending guideline 76/768/EEC, concerning cosmetic products, for the purposes of adapting Annex VII to technical progress), to which reference is explicitly made here.

### Inorganic and Organic Fillers

### Powder Raw Materials/Fillers/Sensory Feel Modifiers

Suitable fillers are talc, silicon oxide, zinc stearate, mica, kaolin, nylon (especially Orgasol) powders, polyethylene powders, polypropylene powders, acrylate powders, teflon, starch, boron nitride, copolymer microspheres such as Expancel (Nobel Industrie), Polytrap (Dow Corning) and silicone resin microbeads (Tospearl from Toshiba).

Other fillers which can be used in the compositions of the invention comprise other inorganic powders such as lime, highly disperse silica, calcium oxide, calcium carbonate, magnesium oxide, magnesium carbonate, Fuller's earth, attapulgite, bentonite, muscovite, phlogopite, synthetic mica, lepidolite, hectorite, biotite, lithium mica, vermiculite, aluminum silicate, aluminum magnesium silicate, diatomaceous earth, starch, alkyl- and/or trialkylarylammonium smectites, chemically modified magnesium aluminum silicate, organically modified montmorillonite, hydrated aluminum silicate, hydrated silica, highly disperse aluminum starch-octenyl succinate, barium silicate, calcium silicate, magnesium silicate, strontium silicate, metal tungsate, magnesium, silicon oxide-aluminum oxide, zeolite, barium sulfate, calcined calcium sulfate (calcined gypsum), calcium phosphate, fluorapatite, hydroxyapatite, ceramic powders, metal soap (zinc stearate, magnesium stearate, zinc myristate, calcium palmitate and aluminum stearate), colloidal silicon dioxide; organic powders, cyclodextrin, methylpolymethacrylate powders, copolymer powders of styrene and acrylic acid, benzoguanamine resin powders and poly(ethylene tetrafluoride) powders; silicon oxide dimethylsilylate, methyl methacrylate cross polymer, silsesquioxane, vinyldimethicone/methicone silsesquioxane crosspolymer, lauroyl lysine.

### Emulsifier

In one embodiment of the invention, the inventive formulations comprise at least one emulsifier.

Every emulsifier is assigned a so-called HLB value (a dimensionless number between 0 and 20) which specifies whether there is a preference for water or oil solubility. Numbers below 9 indicate preferentially oil-soluble, hydrophobic emulsifiers, numbers above 11 water-soluble, hydrophilic emulsifiers. The HLB value says something about the equilibrium of the size and strength of the hydrophilic and lipophilic groups of an emulsifier. The HLB value of an emulsifier can also be calculated from increments, and the HLB increments for the different hydrophilic and hydrophobic groups from which a molecule is composed. It can generally be found in tabular works (e.g. H. P. Fiedler, Lexikon der Hilfsstoffe für Pharmazie, Kosmetik and angrenzende Gebiete [Lexicon of the Excipients for Pharmacy, Cosmetics and Related Fields], Editio Cantor Verlag, Aulendorf, 4th Ed. 1996) or manufacturers' data. The solubility of the emulsifier in the two phases effectively determines the emulsion type. When the emulsifier has better solubility in water, an O/W emulsion is obtained. When the emulsifier, in contrast, has better solubility in the oil phase, a W/O emulsion arises under otherwise identical production conditions.

In one embodiment of the invention, the inventive preparation comprises more than one emulsifier. Depending on the other components, the person skilled in the art uses customary emulsifier systems (for example emulsifier and coemulsifier).

### Nonionic Emulsifiers

The group of nonionic emulsifiers includes, for example:
(1) Addition products of from 2 to 50 mol of ethylene oxide and/or 1 to 20 mol of propylene oxide onto linear fatty alcohols having 8 to 40 carbon atoms, onto fatty acids having 12 to 40 carbon atoms and onto alkylphenols having 8 to 15 carbon atoms in the alkyl group.
(2) C₁₂-C₁₈ fatty acid mono- and diesters of addition products of from 1 to 50 mol of ethylene oxide onto glycerol.
(3) Sorbitan mono- and diesters of saturated and unsaturated fatty acids having 6 to 22 carbon atoms and the ethylene oxide addition products thereof.
(4) Alkyl mono- and oligoglycosides having 8 to 22 carbon atoms in the alkyl radical and ethoxylated analogs thereof.
(5) Addition products of from 7 to 60 mol of ethylene oxide onto castor oil and/or hydrogenated castor oil.
(6) Polyol and especially polyglyceryl esters, for example polyol poly-12-hydroxystearates, polyglyceryl polyricinoleate, polyglyceryl diisostearate or polyglyceryl dimerate. Likewise suitable are mixtures of compounds of two or more of these substance classes.
(7) Addition products of from 2 to 15 mol of ethylene oxide onto castor oil and/or hydrogenated castor oil.
(8) Partial esters based on linear, branched, unsaturated or saturated C₆-C₂₂ fatty acids, ricinoleic acid and 12-hydroxystearic acid and polyglycerol, pentaerythritol, dipentaerythritol, sugar alcohols (e.g. sorbitol), sucrose esters, alkyl glucosides (e.g. methyl glucoside, butyl glucoside, lauryl glucoside) and polyglucosides (e.g. cellulose), or mixed esters, for example glyceryl stearate citrate and glyceryl stearate lactate.
(9) Polysiloxane-polyalkyl-polyether copolymers and corresponding derivatives.
(10) Mixed esters of pentaerythritol, fatty acids, citric acid and fatty alcohol and/or mixed esters of fatty acids having 6 to 22 carbon atoms, methylglucose and polyols, preferably glycerol or polyglycerol.

The addition products of ethylene oxide and/or of propylene oxide onto fatty alcohols, fatty acids, alkylphenols, glycerol mono- and diesters, and also sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. Depending on the degree of ethoxylation, they are W/O or O/W emulsifiers. C_{12/18} fatty acid mono- and diesters of addition products of ethylene oxide onto glycerol are known as refatting agents for cosmetic preparations.

Mild emulsifiers which are particularly suitable in accordance with the invention are polyol poly-12-hydroxystearates and mixtures thereof, which are sold, for example, under the “Dehymuls® PGPH” (W/O emulsifier) or “Eumulgin® VL 75” (blend with Lauryl Glucosides in a weight ratio of 1:1, O/W emulsifier) or Dehymuls® SBL (W/O emulsifier) brands by BASF Personal Care and Nutrition Deutschland GmbH. In this connection, reference may be made especially to European patent EP 766 661 B1. The polyol component of these emulsifiers may derive from substances which have at least two, preferably 3 to 12 and especially 3 to 8 hydroxyl groups and 2 to 12 carbon atoms.

Particularly preferred emulsifiers are, for example, Cetyl Dimethicone Copolyol (e.g. Abil EM-90), Polyglyceryl-2 Dipolyhydroxystearate (e.g. Dehymuls PGPH), Polyglyceryl-3 Diisostearate (e.g. Lameform TGI), Polyglyceryl-4 Isostearate (e.g. Isolan GI 34), Polyglyceryl-3 Oleate (e.g. Isolan GO 33), Diisostearoyl Polyglyceryl-3 Diisostearate (e.g. Isolan PDI), Polyglyceryl-3 Methylglucose Distearate (e.g. Tego Care 450), Polyglyceryl-3 Beeswax (e.g. Cera Bellina), Polyglyceryl-4 Caprate (e.g. Polyglycerol Caprate T2010/90), Polyglyceryl-3 Cetyl Ether (e.g. Chimexane NL), Polyglyceryl-3 Distearate (e.g. Cremophor GS 32) and Polyglyceryl Polyricinoleate (e.g. Admul WOL 1403), Glyceryl Oleate (e.g.

Monomuls 90-0 18), Alkyl Glucoside (e.g. Plantacare 1200, Emulgade PL 68/50, Montanov 68, Tego Care CG 90, Tego Glucosid L 55), Methyl Glucose Isostearate (e.g. Tego Care IS), Methyl Glucose Sesquistearate (Tego Care PS), Sodium Cocoyl Hydrolyzed Wheat Protein (e.g. Gluadin WK), Potassium Cetyl Phosphate (e.g. Amphisol K, Crodafos CKP), Sodium Alkylsulfate (e.g. Lanette E), Sucrose Ester (e.g. Crodesta F-10, F-20, F-50, F-70, F-110, F-160, SL-40, Emulgade® Sucro), ethoxylated and/or propoxylated fatty alcohols, fatty acids, castor oils and hydrogenated castor oils (e.g. Eumulgin B2, B2, B3, L, HRE 40, HRE 60, RO 40, Cremophor HRE 40, HRE 60, L, WO 7, Dehymuls HRE 7, Arlacel 989), PEG-30 Dipolyhydroxystearate (e.g. Arlacel P 135, Dehymuls LE), sorbitan esters, sorbitan esters ethoxylated and/or propoxylated, and mixtures thereof. A particularly effective mixture consists of Polyglyceryl-2 Dipolyhydroxystearate and Lauryl Glucoside and glycerol (e.g. Eumulgin VL 75). Also suitable are Polyglyceryl-4 Diisostearate/Polyhydroxystearate/Sebacate (Isolan® GPS), Diisostearoyl Polyglyceryl-3 Diisostearate (e.g. Isolan PDI), alkali metal salts of acylglutamates (e.g. Eumulgin SG).

Suitable as lipophilic W/O emulsifiers are in principle emulsifiers with an HLB value of 1 to 8, which are summarized in numerous tabular works and are known to those skilled in the art. Some of these emulsifiers are listed, for example, in Kirk-Othmer,“Encyclopedia of Chemical Technology”, 3rd Ed., 1979, Volume 8, page 913. For ethoxylated products, the HLB value can also be calculated according to the following formula: HLB=(100−L): 5, where L is the proportion by weight of lipophilic groups, i.e. the fatty alkyl or fatty acyl groups in percent by weight, in the ethylene oxide adducts.

Particularly advantageous from the group of W/O emulsifiers are partial esters of polyols, especially of C₄-C₆ polyols, for example partial esters of pentaerythritol or sugar esters, e.g. sucrose distearate, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical-grade mixtures thereof. Also suitable as emulsifiers are addition products of 1 to 30 and preferably 5 to 10 mol of ethylene oxide onto the specified sorbitan esters.

Depending on the formulation, it may be advantageous to additionally use at least one emulsifier from the group of nonionic O/W emulsifiers (HLB value: 8-18) and/or solubilizers. These are, for example, the ethylene oxide adducts already mentioned in the introduction and having a correspondingly high degree of ethoxylation, e.g. 10-20 ethylene oxide units for O/W emulsifiers and 20-40 ethylene oxide units for so-called solubilizers. Particularly advantageous O/W emulsifiers in accordance with the invention are Ceteareth-12 and PEG-20 Stearate. Preferred suitable solubilizers are Emulgin® HRE 40 (INCI: PEG-40 Hydrogenated Castor Oil), Eumulgin® HRE 60 (INCI: PEG-60 Hydrogenated Castor Oil), Eumulgin® L (INCI: PPG-1-PEG-9 Lauryl Glycol Ether), and Eumulgin® SML 20 (INCI: Polysorbate-20).

Nonionic emulsifiers from the group of alkyl oligoglycosides are particularly skin-friendly and therefore preferentially suitable as O/W emulsifiers. C₈-C₂₂-alkyl mono- and oligoglycosides, their preparation and their use are known from the prior art. Their preparation takes place in particular by reacting glucose or oligosaccharides with primary alcohols having 8 to 22 carbon atoms. As regards the glycoside radical, either monoglycosides, in which a cyclic sugar radical is glycosidically bonded to the fatty alcohol, or oligomeric glycosides with a degree of oligomerization up to preferably about 8 are suitable. The degree of oligomerization here is a statistical average value which is based on a homolog distribution customary for such technical-grade products. Products which are available under the name Plantacare® comprise a glucosidically bonded C₈-C₁₆-alkyl group onto an oligoglucoside radical whose average degree of oligomerization is 1 to 2. The acylglucamides derived from glucamine are also suitable as nonionic emulsifiers. Preference is given in accordance with the invention to a product which is sold under the name Emulgade® PL 68/50 by BASF Personal Care and Nutrition Deutschland GmbH and is a 1:1 mixture of alkyl polyglucosides and fatty alcohols. Also usable in accordance with the invention is a mixture of Lauryl Glucoside, Polyglyceryl-2 Dipolyhydroxystearate, glycerol and water, which is commercially available under the name Eumulgin® VL 75.

Also advantageously usable in accordance with the invention are sucrose esters such as the mixture of sucrose polystearate and hydrogenated polyisobutene, which is commercially available under the name EMULGADE® Sucro. And also the mixture of sucrose polystearate and cetyl palmitate, which is commercially available under the name EMULGADE® Sucro Plus.

Also suitable as emulsifiers are substances such as lecithins and phospholipids. Examples of natural lecithins include the cephalins, which are also referred to as phosphatidic acids and are derivatives of 1,2-diacyl-sn-glycerol-3-phosphoric acids. In contrast, phospholipids are usually understood to mean mono- and preferably diesters of phosphoric acid with glycerol (glycerol phosphates), which are generally counted among the fats. In addition, sphingosines or sphingolipids are also possible.

Emulsifiers present may, for example, be silicone emulsifiers. These may be selected, for example, from the group of alkylmethicone copolyols and/or alkyldimethicone copolyols, especially from the group of compounds which are characterized by the following chemical structure:

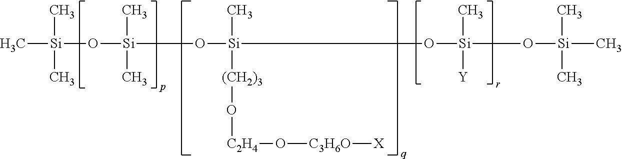

in which X and Y are independently selected from the group of H (hydrogen) and the branched and unbranched alkyl groups, acyl groups and alkoxy groups having 1-24 carbon atoms, p is 0-200, q is 1-40, and r is 1-100.

One example of silicone emulsifiers to be used particularly advantageously within the context of the present invention is that of dimethicone copolyols, which are sold by Evonik Evonik under the trade names AXIL® B 8842, ABIL® B 8843, ABIL® B 8847, ABIL® B 8851, ABIL® B 8852, ABIL® B 8863, ABIL® B 8873 and ABIL®B 88183. A further example of interface-active substances to be used particularly advantageously within the context of the present invention is that of cetyl PEG/PPG-10/1 dimethicone (cetyl dimethicone copolyol), which is sold by Evonik Evonik under the trade name ABIL® EM 90. A further example of interface-active substances to be used particularly advantageously within the context of the present invention is that of cyclomethicone dimethicone copolyol, which is sold by Evonik Evonik under the trade name ABIL®EM 97 and ABIL®WE 09. In addition, the emulsifier Lauryl PEG/PPG-18/18 Methicone (laurylmethicone copolyol) has been found to be very particularly advantageous and is available under the trade name Dow Corning® 5200 Formulation Aid from Dow Corning Ltd. A further advantageous silicone emulsifier is Octyl Dimethicone Ethoxy Glucoside from Wacker.

For an inventive water-in-silicone oil emulsion, it is possible to use all emulsifiers known for this type of emulsion. Water-in-silicone oil emulsifiers that are particularly preferred in accordance with the invention here are Cetyl PEG/PPG 10/1 Dimethicone and Lauryl PEG/PPG-18/18 Methicone [e.g. ABIL® EM 90 (Evonik Evonik), DC5200 Formulation Aid (Dow Corning)] and any desired mixtures of the two emulsifiers.

### Surfactants

In one embodiment of the invention, the inventive preparations comprise at least one surfactant.

Surfactants are amphiphilic substances which can dissolve organic, nonpolar substances in water. As a result of their specific molecular structure having at least one hydrophilic molecular moiety and one hydrophobic molecular moiety, they are able to reduce the surface tension of water, wet the skin, facilitate the removal and dissolution of dirt, facilitate rinsing and—if desired—control foaming.

Surfactants are typically understood to mean surface-active substances having an HLB value of greater than 20.

Surface-active substances present may be anionic, nonionic, cationic and/or amphoteric or zwitterionic surfactants. At least one anionic surfactant is preferably present in surfactant-containing cosmetic preparations, such as, for example, shower gels, foam baths, shampoos, etc.

The preparations according to the invention comprise the surfactant(s) typically in an amount of 0 to 40% by weight, preferably 0.05 to 30% by weight, especially 0.05 to 20% by weight, preferably 0.1 to 15% by weight and especially 0.1 to 10% by weight, based on the total weight of the preparation.

Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partially oxidized alk(en)yl oligoglycosides and glucuronic acid derivatives, fatty acid N-alkylglucamides, protein hydrolyzates (especially wheat-based vegetable products), polyol fatty acid esters, polyglycerol fatty acid esters, sugar esters, sorbitan esters, polysorbates and amine oxides, polyether-modified polysiloxanes, polyether-modified siloxane elastomers. If the nonionic surfactants comprise polyglycol ether chains, these may have a conventional homolog distribution, but preferably have a narrowed homolog distribution.

Zwitterionic surfactants is the term used for those surface-active compounds which bear at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazoline having in each case 8 to 18 carbon atoms in the alkyl or acyl group, and also cocoacylaminoethyl hydroxyethylcarboxymethylglycinate. A preferred zwitterionic surfactant is the fatty acid amide derivative known under the INCI name Cocamidopropyl Betaine.

Likewise suitable, especially as cosurfactants, are ampholytic surfactants. Ampholytic surfactants are understood to mean those surface-active compounds which, apart from a C₈-C₁₈-alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkylimino-dipropionic acids, N-hydroxyethyl-N-alkylamidopropylglycines, N-alkyltaurines, N-alkylsarcosines, 2-alkylaminopropionic acids and alkylaminoacetic acids each having about 8 to 18 carbon atoms in the alkyl group. Particularly preferred ampholytic surfactants are N-cocoalkylaminopropionate, cocoacylaminoethylaminopropionate and C₁₂₋₁₈- acylsarcosine.

Typical examples of amphoteric or zwitterionic surfactants are alkyl betaines, alkyl amido betaines, aminopropionates, aminoglycinates, imidazolinium betaines and sulfobetaines. The surfactants mentioned are exclusively known compounds. With regard to the structure and preparation of these substances, reference may be made to relevant review works in this field. Typical examples of particularly suitable mild, i.e. particularly skin-friendly, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ethercarboxylic acids, alkyl oligoglucosides and/or mixtures thereof with alkyl oligoglucoside carboxylates, fatty acid glucamides, alkylamidobetaines, amphoacetals and/or protein fatty acid condensates, the latter preferably based on wheat proteins or salts thereof.

Anionic surfactants are characterized by a water-solubilizing, anionic group, for example a carboxylate, sulfate, sulfonate or phosphate group and a lipophilic radical. Skin-compatible anionic surfactants are known to the person skilled in the art in a large number from relevant handbooks and are commercially available. These are especially alkyl sulfates in the form of their alkali metal, ammonium or alkanolammonium salts, alkyl ether sulfates, alkyl ether carboxylates, acyl isethionates, acyl sarcosinates, acyl taurines with linear alkyl or acyl groups having 12 to 18 carbon atoms, and also sulfosuccinates and acyl glutamates in the form of their alkali metal or ammonium salts.

Typical examples of anionic surfactants are soaps, alkali metal (e.g. sodium and potassium) salts, ammonium and alkylammonium salts, alkylbenzenesulfonates, alkanesulfonates, olefin-sulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ethercarboxylic acids and salts thereof, fatty acid isethionates, fatty acid sarcosinates, fatty acid taurides, alkyl lactates, alkyl tartrates, alkyl citrates, alkyl phosphates, N-acylamino acids, for example acyl glutamates and acyl aspartates, alkyl oligoglucoside sulfates, protein fatty acid condensates (especially vegetable products based on wheat) and alkyl (ether) phosphates. If the anionic surfactants comprise polyglycol ether chains, these may have a conventional homolog distribution, but preferably have a narrowed homolog distribution.

Usable cationic surfactants are especially quaternary ammonium compounds. Preference is given to ammonium halides, especially chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. In addition, the very readily biodegradable quaternary ester compounds, for example the dialkylammonium methosulfates and methylhydroxyalkyldialkoyloxyalkylammonium methosulfates sold under the trade name Stepantex® and the corresponding products of the Dehyquart® series can be used as cationic surfactants. The term “ester quats” is generally understood to mean quaternized fatty acid triethanolamine ester salts. They can impart an exceptional soft feel to the inventive preparations. These are known substances which are prepared by the relevant methods of organic chemistry. Further cationic surfactants usable in accordance with the invention are the quaternized protein hydrolyzates.

### Wax Component

In one embodiment of the invention, the inventive preparations comprise at least one wax component.

The preparations according to the invention comprise the wax component(s) typically in an amount of 0 to 40% by weight, especially 0 to 20% by weight, preferably 0.1 to 15% by weight and especially 0.1 to 10% by weight, based on the total weight of the preparation.

The term “wax” is typically understood to mean all natural or synthetic substances and substance mixtures having the following properties: they are of solid to brittle and hard consistency, coarse to finely crystalline, transparent to cloudy and melt above 30° C. without decomposition. They are low in viscosity even a little above the melting point and do not string, and exhibit a strongly temperature-dependent consistency and solubility. It is possible in accordance with the invention to use a wax component or a mixture of wax components which melt at 30° C. or higher.

The waxes used in accordance with the invention may also be fats and fat-like substances with waxy consistency, provided they have the required melting point. These include, inter alia, fats (triglycerides), mono- and diglycerides, natural and synthetic waxes, fatty and wax alcohols, fatty acids, esters of fatty alcohols and fatty acids and also fatty acid amides or any desired mixtures of these substances.

Fats are understood to mean triacylglycerols, i.e. the triple esters of fatty acids with glycerol. They preferably comprise saturated, unbranched and unsubstituted fatty acid radicals. They may also be mixed esters, i.e. triple esters of glycerol with different fatty acids. Usable in accordance with the invention and of particularly good suitability as consistency regulators are so-called hydrogenated fats and oils, which are obtained by partial hydrogenation. Vegetable so-called hydrogenated fats and oils are preferred, e.g. hydrogenated castor oil, peanut oil, soybean oil, rapeseed oil, colza oil, cottonseed oil, soybean oil, sunflower oil, palm oil, palm kernel oil, linseed oil, almond oil, corn oil, olive oil, sesame oil, cocoa butter and coconut fat.

Suitable examples include the triple esters of glycerol with C12-C60-fatty acids and especially C12-C36-fatty acids. These include hydrogenated castor oil, a triple ester of glycerol and a hydroxystearic acid, which is commercially available, for example, under the Cutina HR name. Glyceryl tristearate, glyceryl tribehenate (e.g. Syncrowax HRC), glyceryl tripalmitate or the triglyceride mixtures known under the Syncrowax HGLC name are likewise suitable, with the proviso that the melting point of the wax component or of the mixture is 30° C. or higher.

Wax components usable in accordance with the invention are especially mono- and diglycerides and mixtures of these partial glycerides. Glyceride mixtures usable in accordance with the invention include the Novata AB and Novata B (mixture of C12-C18-mono-, -di- and -triglycerides) and Cutina MD or Cutina GMS (glyceryl stearate) products sold by BASF Personal Care and Nutrition Deutschland GmbH & Co. KG.

Fatty alcohols usable in accordance with the invention as the wax component include the C12-C50-fatty alcohols. The fatty alcohols can be obtained from natural fats, oils and waxes, for example myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. Preference is given in accordance with the invention to saturated unbranched fatty alcohols. However, it is also possible in accordance with the invention to use unsaturated, branched or unbranched fatty alcohols as the wax component, provided they have the required melting point. It is also possible in accordance with the invention to use fatty alcohol cuts, as produced in the reduction of naturally occurring fats and oils, for example bovine tallow, peanut oil, colza oil, cottonseed oil, soybean oil, sunflower oil, palm kernel oil, linseed oil, castor oil, corn oil, rapeseed oil, sesame oil, cocoa butter and coconut fat. However, it is also possible to use synthetic alcohols, e.g. the linear, even-numbered fatty alcohols from the Ziegler synthesis (alfols) or the partially branched alcohols from the oxo process (dobanols). Particular preferably suitable in accordance with the invention are C14-C22-fatty alcohols, which are sold, for example, by BASF Personal Care and Nutrition Deutschland GmbH under the Lanette 18 (C18-alcohol), Lanette 16 (C16-alcohol), Lanette 14 (C14-alcohol), Lanette 0 (C16/C18-alcohol) and Lanette 22 (C18/C22-alcohol) names. Fatty alcohols impart a drier skinfeel to the preparations than triglycerides and are therefore preferred over the latter.

The wax components used may also be C14-C40-fatty acids or mixtures thereof. These include, for example, myristic acid, pentadecanoic acid, palmitic acid, margaric acid, stearic acid, nonadecanoic acid, arachic acid, behenic acid, lignoceric acid, cerotic acid, melissic acid, erucic acid and eleostearic acid, and also substituted fatty acids, for example 12-hydroxystearic acid, and the amides or monoethanolamides of the fatty acids, this list being illustrative and nonlimiting in character.

It is possible in accordance with the invention to use, for example, natural vegetable waxes, such as candelilla wax, carnauba wax, japan wax, esparto grass wax, cork wax, guaruma wax, rice germ wax, sugarcane wax, ouricury wax, montan wax, sunflower wax, fruit waxes such as orange waxes, lemon waxes, grapefruit wax, bayberry wax, and animal waxes, for example beeswax, shellac wax, spermaceti, wool wax and uropygial grease. In the context of the invention, it may be advantageous to use hydrogenated or hardened waxes. The natural waxes usable in accordance with the invention also include mineral waxes, for example ceresin and ozokerite or the petrochemical waxes, for example petrolatum, paraffin waxes and microwaxes. Usable wax components also include chemically modified waxes, especially the hard waxes, for example montan ester waxes, sasol waxes and hydrogenated jojoba waxes. Synthetic waxes usable in accordance with the invention include, for example, wax-like polyalkylene waxes and polyethylene glycol waxes. Vegetable waxes are preferred in accordance with the invention.

The wax component can likewise be selected from the group of the wax esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (e.g. 12-hydroxystearic acid) and saturated and/or unsaturated, branched and/or unbranched alcohols, and also from the group of lactides of long-chain hydroxycarboxylic acids.

Examples of such esters are the C16-C40-alkyl stearates, C20-C40-alkyl stearates (e.g. Kesterwachs K82H), C20-C40-dialkyl esters of dimeric acids, C18-C38-alkylhydroxystearoyl stearates or C20-C40-alkyl erucates. It is also possible to use C30-C50-alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate. Fatty acid partial glycerides, i.e. technical-grade mono- and/or diesters of glycerol with fatty acids having 12 to 18 carbon atoms, for example glycerol mono/dilaurate, -palmitate, -myristate or stearate, are also useful for this purpose.

Suitable waxes are additionally pearlescent waxes. Possible pearlizing waxes, in particular for use in surface-active formulations, are, for example: alkylene glycol esters, especially ethylene glycol distearate; fatty acid alkanolamides, especially coconut fatty acid diethanolamide; partial glycerides, especially stearic acid monoglyceride; esters of polyvalent, optionally hydroxy-substituted, carboxylic acids with fatty alcohols having 6 to 22 carbon atoms, especially long-chain esters of tartaric acid; fatty substances, such as, for example, fatty alcohols, fatty ketones, fatty aldehydes, fatty ethers and fatty carbonates, which have in total at least 24 carbon atoms, especially laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

### Oil Bodies

The oil and/or wax in the textured composition according to the invention is selected from the group comprising fatty acid esters, Guerbet alcohols, tri- or partial glycerides, mono-/dialkyl ethers, mono-/dialkyl carbonates, oil-soluble UV filters, fatty alcohol ethers, microcrystalline waxes, hydrocarbons or mineral oil, silicone oil, natural vegetable oils and mixtures thereof.

Preferred oils and/or waxes are Guerbet alcohols based on fatty alcohols having 6 to 18, preferably 8 to 10 carbon atoms (Eutanol G, Eutanol G 16), esters of linear C₆-C₂₂-fatty acids with linear or branched C₆-C₂₂-fatty alcohols or esters of branched C₆-C₁₃-carboxylic acids with linear or branched C₆-C₂₂-fatty alcohols such as, e.g. myristyl myristate (Cetiol® MM), myristyl palmitate, myristyl stearate, myristyl isostearate, myristyl oleate, myristyl behenate, myristyl erucate, cetyl myristate, cetyl palmitate (Cutina® CP), cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, isopropyl myristate, isopropyl palmitate, oleyl myristate, oleyl palmitate, oleyl stearate, oleyl isostearate, oleyl oleate, oleyl behenate, oleyl erucate (Cetiol® J 600), behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate, ethylhexyl stearate (Cetiol® 868), hexyl laurate (Cetiol® A), C12.15 alkyl benzoate (Cetiol® AB), dibutyl adipate (Cetiol® B), coco-caprylate (Cetiol® C5), coco-caprylate/caprate (Cetiol® LC, Cetiol® C 5C), propylheptyl caprylate (Cetiol® Sensoft), cetearyl isononanoate (Cetiol® SN), decyl oleate (Cetiol® V), cetearyl ethylhexanoate (Luvitol® EHO), also suitable are esters of C₁₈-C₃₈-alkylhydroxycarboxylic acids with linear or branched C₆-C₂₂-fatty alcohols, especially dioctyl malate, esters of linear and/or branched fatty acids with polyhydric alcohols (e.g. propylene glycol, dimerdiol or trimertriol) such as propylene glycol dicaprylate/dicaprate (Myritol® PGDC) and/or Guerbet alcohols, triglycerides based on C₆-C₁₀-fatty acids, liquid mono-/di-/triglyceride mixtures based on C₆-C₁₈-fatty acids (Myritol® 331, Myritol® 312, Myritol® 318), esters of C₆-C₂₂-fatt alcohols and/or Guerbet alcohols with aromatic carboxylic acids, especially benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatt alcohol carbonates, e.g. dicaprylyl carbonate (Cetiol® CC), Guerbet carbonates based on fatty alcohols having 6 to 18, preferably 8 to 10 carbon atoms, esters of benzoic acid with linear and/or branched C₆-C₂₂-alcohols (e.g. Finsolv® TN, Cetiol® AB), linear or branched, symmetrical or asymmetrical dialkyl ethers having 6 to 22 carbon atoms per alkyl group, e.g. dicaprylyl ether (Cetiol® OE), ring-opening products of epoxidized fatty acid esters with polyols (Cetiol E). Further suitable emollients are vegetable oils (Cegesoft® GPO, Cegesoft® PFO, Cegesoft® PS 6, Cegesoft® SBE, Cegesoft® SH) and mixtures thereof (Cegesoft® VP), silicone oils, hydrocarbons such as mineral oils, isoparaffins, paraffinum liquidum, undecane/tridecane (Cetiol® Ultimate), hydrogenated polyisobutenes (Luvitol® Light), mineral oils, isoparaffins, paraffins.

Suitable further oil bodies are, for example, Guerbet alcohols based on fatty alcohols having 6 to 18, preferably 8 to 10, carbon atoms, and also further additional esters such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C₁₈-C₃₈-alkylhydroxycarboxylic acids with linear or branched C₆-C₂₂-fatty alcohols, especially dioctyl malate, esters of linear and/or branched fatty acids with polyhydric alcohols (e.g. propylene glycol, dimerdiol or trimertriol), triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, especially benzoic acid, esters of C₂-C₁₂-dicarboxylic acids with polyols having 2 to 10 carbon atoms and 2 to 6 hydroxyl groups, vegetable oils, branched primary alcohols, substituted cyclohexanes, linear and branched C₆-C₂₂-fatty alcohol carbonates, e.g. dicaprylyl carbonate (Cetiol® CC), Guerbet carbonates based on fatty alcohols having 6 to 18, preferably 8 to 10 carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as dicaprylyl ether (Cetiol® OE), ring-opening products of epoxidized fatty acid esters with polyols.

Useful further oil bodies are, for example, silicone oils. They may be present as cyclic and/or linear silicone oils. Silicone oils are high molecular weight synthetic polymeric compounds in which silicon atoms are joined via oxygen atoms in a chain-like and/or grid-like manner and the remaining valences of silicon are satisfied by hydrocarbon radicals (usually methyl, more rarely ethyl, propyl, phenyl groups etc.). Systematically, the silicone oils are referred to as polyorganosiloxanes. The methyl-substituted polyorganosiloxanes, which are the most important compounds of this group in terms of volume and are characterized by the structural formula below

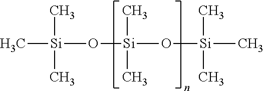

are also referred to as Polydimethylsiloxane or Dimethicone (INCI). Dimethicones come in various chain lengths and with various molecular weights.

Advantageous polyorganosiloxanes in the context of the present invention are, for example, dimethylpolysiloxane [poly(dimethylsiloxane)], which are available, for example, under the Abil 10 to 10 000 trade names from Evonik Evonik. Also advantageous are phenylmethylpolysiloxane (INCI: Phenyl Dimethicone, Phenyl Trimethicone), cyclic silicones (octamethylcyclotetrasiloxane or decamethylcyclopentasiloxane), which are also referred to according to INCI as Cyclomethicone, aminomodified silicones (INCI: Amodimethicone) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are avaibale from Evonik Evonik as various Abil wax types. However, other silicone oils are also to be used advantageously for the purposes of the present invention, for example cetyldimethicone, hexamethylcyclotrisiloxane, polydimethylsiloxane, poly(methylphenylsiloxane). Silicones which are particularly preferred in accordance with the invention are dimethicone and cyclomethicone.

The preparations according to the invention may also comprise preservatives, perfume oils, superfatting agents, stabilizers and/or hydrotropes. Suitable preservatives are, for example, phenoxyethanol, formaldehyde solution, parabens, pentanediol or sorbic acid, benzoic acid and salts thereof, benzyl alcohol, benzyl salicylate. Dehydroacetic acid, methylthiazolinone or sorbic acid and salts thereof, and also the silver complexes known under the name Surfacine®, and the additional substance classes listed in Annex 6, parts A and B, of the Cosmetics Directive. Substances are also used which function as preservative aids such as ethylhexylglycerin and caprylyl glycol and also polyols and alcohols such as propanediol, phenylpropanol, phenethyl alcohol and undecyl alcohol and also the silver complexes known by the name Surfacine®. Additionally suitable as preservatives are the 1,2-alkanediols having 5 to 8 carbon atoms, which are described in WO 07/048757.

Suitable preservatives are especially the substances approved according to Annex VI of the Commission Directive (in the version of the EU guideline 2005/9/EC of Jan. 28, 2005 amending guideline 76/768/EEC, concerning cosmetic products, for the purposes of adapting Annex VII to technical progress), to which reference is explicitly made here.

Perfume oils include mixtures of natural and synthetic odorants. Natural odorants are extracts of flowers, stems and leaves, fruit, fruit shells, roots, wood, herbs and grasses, needles and branches, resins and balsams. Additionally possible are animal raw materials, such as, for example, civet and castoreum, and also synthetic odorant compounds of the ester, ether, aldehyde, ketone, alcohol and hydrocarbon type.

Stabilizers that may be mentioned are metal salts of fatty acids, for example magnesium, aluminum and/or zinc stearate or ricinoleate and also complexing agents, also chelating agents. They are chemical compounds which form chelate complexes with metal ions. They are used for the purposes of masking (undesirable) chemical properties of metal ions, e.g. tetrasodium iminodisuccinate (also: iminodisuccinate tetrasodium salt), ethylenediaminetetraacetic acid or ethylenediamine tetraacetate.

To improve the flow characteristics, it is also possible to use hydrotropes, for example ethanol, isopropyl alcohol, or polyols. Polyols which are suitable here preferably have 2 to 15 carbon atoms and at least two hydroxyl groups. The polyols may comprise still other functional groups, in particular amino groups, or be modified with nitrogen.

### Examples

### A1: Test Methods for Testing the Anti-Pollution Effect

In order to test whether substances have an anti-pollution effect on human skin, especially protection of human skin from dust, especially fine dust, the test method described below was employed.

Activated carbon (obtained from Carl Roth GmbH, Karlsruhe, Germany) was used as protection model. Activated carbon is microcrystalline carbon. There is a difference in size between the particles of activated carbon and the particles of fine dust. Nevertheless, activated carbon is a suitable model substance for fine dust. Activated carbon is also suitable for investigations of human subjects since it is toxicologically safe compared to many other substances.

A test setup was developed for the method in order to be able to investigate the anti-adhesive effect on the skin of the substances to be tested. The apparatus used in this case served the defined application of activated carbon to the underarms of human subjects. In this case, a defined amount of activated carbon was placed in a chamber and, under standardized pressure, applied to the skin of the underarm via an air stream.

The test substance was applied to the skin precleansed with ethanol. For this purpose, a standardized amount of the test substance was applied to a defined area of the skin of the underarm (if not stated otherwise below, this was 2 mg/cm²). Firstly, prior to the treatment, each skin site was recorded by a photographic recording. After application of the test substance, a further photograph of the same site was taken. After a drying time of 10 min, the activated carbon was applied by a blast of air. After treatment with activated carbon, a further photograph was recorded. These images served to determine the amount of dirt (activated carbon) adhering to the skin. This process was carried out for each test substance on four different sites of the underarm.

After the specified measurements on all four sites of the underarm had been carried out, the differences in the ability of the adhering dirt (activated carbon) to be washed off were tested. For this purpose, a defined amount of water was applied to the respective skin sites.

Consistent light conditions were ensured when recording the photographs. Comparison of the treated areas was conducted by means of a suitable computer program. For this purpose, the average grayscale value was taken as a baseline. The value of the skin with adhering activated carbon was correspondingly subtracted from the value of the same skin site without activated carbon. The higher the difference in value obtained, the more activated carbon had stuck to the skin. Subsequently, the average value and the standard deviation of all subjects (if not stated otherwise below, this was 11), was calculated per subject. The ability to be washed off was determined by subtracting the average grayscale value after washing off from that prior to the washing off. The results were stated relative to the results of untreated skin.

### A1-1: Test Methods for Testing the Anti-Pollution Effect on Hair

In a similar process, the effect on hair was investigated: individual hairs were treated with a test substance and were then exposed to a defined amount of activated carbon via an air stream in a chamber using an appropriate apparatus. The individual hairs were photographed with a digital microscope and the degree of soiling was quantified by image evaluation. The individual hairs were rinsed with a defined amount of water and photographed again by digital microscopy and the corresponding soiling determined.

### A2: Method for Determining the Glass Transition Temperature Tg

The glass transition temperature Tg was determined as follows. Polymers, which were present as aqueous dispersions, were freeze-dried prior to measurement. Like all other polymers, they were then subjected to a drying process in the DSC instrument used (TA Instruments DSC Q100). The samples were subjected to an isothermal run at 80° C. for 1 hour. After this run, further 30 minute isothermal runs were carried out until a constant weight of the sample was attained. The determination of the polymer glass transition temperatures Tg was conducted with the same instrument in MDSC mode (DSC measurement). Standard aluminum pans which comprised 2 to 10 mg of the dried polymer were heated in the temperature range of −50° C. to 200° C. with a heating rate of 1K/min and a modulation rate of 0.5K/min. The reversible heating signal was evaluated according to the standard “glass/step transition” of the “Universal Analysis 2000” software (Version 4.7A, TA Instruments, Eschborn/Germany). The glass transition temperature was specified as the inflection point of the curve.

### B: Substances Tested

Polymers 1 to 9 described below were tested. % signifies % by weight.

### First Group: Polymers or Copolymers of Acrylic Acid or of Methacrylic Acid

| Polymer No. | Name | More accurate designation |
|---|---|---|
| Polymer 1 | Carbomer | Acrylic acid, crosslinked (with |
| | (INCI | less than 1% by weight crosslinker) |
| | name for | |
| | polyacrylic | |
| | acid) | |
| Polymer 2 | An acrylate | A copolymer composed of ethyl acrylate |
| | copolymer | (30-60%), methacrylic acid (30-60%), |
| | | methacrylamide (5-30%), C18-alkyl-(EO)25 |
| | | methacrylate (0.1-10%), pentaerythritol |
| | | triallyl ether (0.01-1.0%) |
| Polymer 3 | An acrylate | A copolymer composed of ethyl acrylate |
| | copolymer | (30-60%) and methacrylic acid (40-70%) |
| Polymer 3a | An acrylate | A copolymer composed of ethyl acrylate |
| | copolymer | (40-60%), methacrylic acid (30-50%), acrylic |
| | | acid (5-15%), pentaerythritol triallyl ether |
| | | (0.01-1.0%) |
| Polymer 4 | An acrylate | A terpolymer composed of tert-butyl acrylate |
| | copolymer | (60-80%), ethyl acrylate (5-20%) and |
| | | methacrylic acid (10-30%) |

Note on the designation: “Acrylate” is understood to mean acrylic acid and methacrylic acid and salts and esters thereof

### Second Group: Cationic Polymers

| Polymer No. | Name | More accurate designation |
|---|---|---|
| Polymer 5 | Polyquaternium- | A copolymer composed of |
| | 68 (INCI) | vinylpyrrolidone (40-65%), |
| | | methacrylamide (25-35%), |
| | | vinylimidazole (5-20%) and quaternary |
| | | (methylated) vinylimidazole (5-20%) |

### Third Group: Caprolactam-Containing Polymers

| Polymer No. | Name | More accurate designation |
|---|---|---|
| Polymer 6 | Polyvinylcaprolactam | N-Vinylcaprolactam |
| | | homopolymer |

### Fourth Group: Copolymers of Vinylpyrrolidone (VP), Vinylimidazole (VI) and Optional Further Monomers

| Polymer No. | Name | More accurate designation |
|---|---|---|
| Polymer 7 | VP/methacrylamide/ | A copolymer composed of |
| | VI copolymer | vinylpyrrolidone (50-70%), |
| | | methacrylamide (20-40%) and |
| | | vinylimidazole (1-10%) |

### Comparative Group: Polycarbonates and Polyurethanes

| Polymer No. | Name | More accurate designation |
|---|---|---|
| Polymer 8 | Hydrogenated dimer | Hydrogenate dimer dilinoleyl/ |
| | dilinoleyl/dimethyl | dimethyl carbonate copolymer |
| | carbonate | |
| | copolymer | |
| Polymer 9 | Polyurethane-39 | Polyurethane |
| | (INCI) | |

### C: Properties of the Polymers Tested and Test Results

(Anti-adhesion/washability measured with samples with 1% by weight polymer in water):

| | | | | Anti-adhesive | Fewer particulate |
|---|---|---|---|---|---|
| | | Average | | effect | substances |
| | | molar mass | | compared to | on the skin |
| | Solubility | at least | Glass | untreated | after washing |
| | at least | 100000 g/mol | transition | skin (at least | (at least 5%) |
| Polymer | 0.1 g/100 g | (weight | temperature | 5% lower | compared to |
| Number | of water | average Mw) | above 70° C. | adhesion) | untreated skin |
| 1 | yes | yes | yes | yes | yes |
| 2 | yes | yes | yes | yes | yes |
| 3 | yes | yes | yes | yes | yes |
| 3a | yes | yes | yes | yes | yes |
| 4 | yes | yes | yes | yes | yes |
| 5 | yes | yes | yes | yes | yes |
| 6 | yes | yes | yes | yes | yes |
| 7 | yes | yes | yes | yes | yes |
| 8 | no | no | no | no | no |
| 9 | yes | no | no | no | no |

These results show that polymers 1 to 7 bring about the desired anti-pollution effect, but polymers 8 and 9 do not. It can be assumed that those polymers that effect protection from dust, also effect continuous and general protection from air pollution, not only from dust, but even from other substances which constitute air pollution.

### C-1: Test Results on Hair

| | Anti-adhesive effect compared | Fewer particulate |
|---|---|---|
| | to untreated hair (at least 5% | substances on the hair |
| Polymer | lower adhesion in comparison | after rinsing (at least 5%) |
| Number | to untreated hair) | compared to untreated hair |
| 2 | no | yes |
| 3 | yes | yes |
| 5 | yes | yes |
| 9 | no | no |

These results show that polymers 3 to 5 bring about the desired anti-pollution effect even on hair, but not the polymer 9.

### D: Formulations

The following formulations show by way of example how cosmetic products may be composed, comprising the aforementioned polymers, and thus should bring about an anti-pollution effect.

The numerical figures refer to % by weight.

Sometimes the polymers are introduced into the formulation already as a solution or in other non-pure form. In these case, the active content of polymer is additionally stated.

### Formulation 1: Semisolid Gel Gel (Cream), Formulation 2-7: Low-Viscosity to Free-Flowing Gel Lotions

| | Chemical name/ | | | | | | | |
|---|---|---|---|---|---|---|---|---|
| Constituents | compostion (%) | 1 | 2 | 3 | 4 | 5 | 6 | 7 |
| Polymer 1 | | 1.00 | | | | | | |
| Polymer 2 | | | 3.30 (= 1.0 | | | | | |
| | | | active | | | | | |
| | | | content) | | | | | |
| Polymer 3 | | | | 3.30 (= 1.0 | | | | |
| | | | | active | | | | |
| | | | | content) | | | | |
| Polymer 4 | | | | | 1.00 | | | |
| Polymer 5 | | | | | | 5.00 (= 1.0 | | |
| | | | | | | active | | |
| | | | | | | content) | | |
| Polymer 6 | | | | | | | 2.50 (= 1.0 | |
| | | | | | | | active | |
| | | | | | | | content) | |
| Polymer 7 | | | | | | | | 5.00 (= 1.0 |
| | | | | | | | | active |
| | | | | | | | | content) |
| Demin. water | Water | to 100 | to 100 | to 100 | to 100 | to 100 | to 100 | to 100 |
| Cetiol ® | Unciecan (and) | 2.50 | | 1.00 | | | 0.50 | |
| Ultimate | triciecane | | | | | | | |
| Cyclomethicone | Cyclomethicone | | 1.00 | | 1.00 | | 0.50 | |
| Tocopherol | Tocopherol | 0.10 | | | | 0.20 | | 0.50 |
| | (Vitamin E) | | | | | | | |
| Cegesoft ® | Olus Oil | | | 0.2 | | | 0.1 | |
| PS 6 | | | | | | | | |
| Tinosorb ® M | Methylen Bis- | 5.00 | | | | 10.00 | | |
| | Benzotriazolyl | | | | | | | |
| | Tetramethylbutyl- | | | | | | | |
| | phenol | | | | | | | |
| | (and) Aqua | | | | | | | |
| | (and) Decyl | | | | | | | |
| | Glucoside (and) | | | | | | | |
| | Propylene Glycol | | | | | | | |
| | (and) Xanthan Gum | | | | | | | |
| Z-Cote ® HP1 | Zinc Oxide (nano), | 1.00 | | 1.00 | | | 4.00 | |
| | Triethoxycaprylylsilane | | | | | | | |
| T-80AS | Titanium Dioxide, | | 10.00 | 5.00 | 10.00 | | 2.00 | |
| | silica, triethoxy | | | | | | | |
| | caprylylsilane | | | | | | | |
| D-Panthenol | Panthenol | | | 0.5% | | | | |
| USP | | | | | | | | |
| Glycerol | Glycerol | 5.00 | 1.00 | 5.00 | 1.00 | 0.5 | 1.00 | |
| PatcH2O ® | Water (and) glycerol | 3.00 | | | | 1.5 | | 3.00 |
| | (and) trehalose | | | | | | | |
| | (and) urea (and) | | | | | | | |
| | serine (and) | | | | | | | |
| | pentylene glycol | | | | | | | |
| | (and) glyceryl | | | | | | | |
| | polyacrylate (and) | | | | | | | |
| | algin (and) caprylyl | | | | | | | |
| | glycol (and) sodium | | | | | | | |
| | hyaluronate (and) | | | | | | | |
| | pullulan (and) | | | | | | | |
| | disodium phosphate | | | | | | | |
| | (and) potassium | | | | | | | |
| | phosphate | | | | | | | |
| Hydagen ® GG | Glyceryl glucoside | | | | | | 5.00 | 12.00 |
| | and glycerol | | | | | | | |
| PURISOFT | Water (and) glycerol | | 3.00 | | | 3.00 | | 3.00 |
| POE LS 9726 | (and) Moringa | | | | | | | |
| | pteryosperma seed | | | | | | | |
| | extract | | | | | | | |
| Talc FM SSA | Talc (and) | 5.00 | | | | 1.00 | | |
| (K.S. Pearl) | dimethicone | | | | | | | |
| Mearlmica ® | Mica, lauroyl lysine | | | 5.00 | | | | 1.00 |
| treated SVA | | | | | | | | |
| Pearl-Glo ® SF | Bismuth oxychloride | | 5.00 | | 5.00 | | | |
| PG1099 | | | | | | | | |
| SA-SB-300 | Silica | | | | | 2.00 | | 1.00 |
| (Miyoshi | | | | | | | | |
| kasei) | | | | | | | | |
| Dow Corning | Dimethicone/vinyl | | | | | 2.00 | | 1.00 |
| 9701 | dimethicone | | | | | | | |
| Cosmetic | crosspolymer (and) | | | | | | | |
| powder (Dow | Silica | | | | | | | |
| Corning) | | | | | | | | |
| STR-100A-LP | Titanium dioxide | | | | | | 5.00 | |
| (Sakai | (nano), hydrated | | | | | | | |
| Chemical | silica, dimethicone/ | | | | | | | |
| Industry) | methicone | | | | | | | |
| | copolymer, | | | | | | | |
| | aluminum hydroxide | | | | | | | |
| Timica ® Terra | Mica, titanium | 1.00 | 1.00 | 0.5 | 1.00 | 0.5 | 0.5 | 1.00 |
| White | dioxide | | | | | | | |
| MN4501 | | | | | | | | |
| Timica ® Terra | Mica, iron oxides, | 0.1 | | 0.1 | | | | |
| Brown | titanium dioxide | | | | | | | |
| MN4509 | | | | | | | | |
| Timic ® Terra | Mica, iron oxides, | | 0.1 | 0.1 | 0.1 | | 0.1 | |
| Yellow | titanium dioxide | | | | | | | |
| MN4502 | | | | | | | | |
| Flamenco ® | Mica, titanium | | | | | | 0.5 | 1.00 |
| Silk Blue | dioxide | | | | | | | |
| 630M | | | | | | | | |
| KOH 20% | Potassium | | to adjust to pH 6.5-7.0 | | | | | |
| | hydroxide | | | | | | | |
| Euxyl ® K 320 | 2-Phenoxyethanol, | | 1.00 | | 1.00 | 1.30 | 1.30 | 1.00 |
| | methyl 4- | | | | | | | |
| | hydroxybenzoate, | | | | | | | |
| | ethyl 4- | | | | | | | |
| | hydroxybenzoate | | | | | | | |
| Protectol ® PE | Phenoxyethanol | 1.00 | | 1.00 | | | | |
| Sensive ® SC | Ethylhexylglycerin | 0.5 | | 0.50 | | | | |
| 50 | | | | | | | | |
| pH | | | 6.8 | 6.6 | 6.5 | 6.4 | 6.5 | 6.6 |

### Formulations 8-13: Gel Lotions that can be Applied by Pump Spray and Aerosol Spray as Skin Protection Agent and/or as Top Coat and/or Base Coat for Additive Use in Skin Care Preparations

| | Chemical name/ | | | | | | |
|---|---|---|---|---|---|---|---|
| Constituents | compostion (%) | 8 | 9 | 10 | 11 | 12 | 13 |
| Polymer 3 | | 3.30 (= 1.0 | 1.65 (= 0.5 | 2.20 | 0.33 (= 0.1 | 1.65 (= 0.5 | 0.33 (= 0.1 |
| | | active | active | | active | active | active |
| | | content) | content) | | content) | content) | content) |
| Tinovis ® | Hydrophobically | | | 0.33 (= 0.1 | | 0.33 (= 0.1 | 0.33 (= 0.1 |
| GTC UP | modified copolymers | | | active | | active | active |
| | of acrylic acid | | | content) | | content) | content) |
| | Acrylate/Beheneth-25 | | | | | | |
| | Methacrylate | | | | | | |
| | Copolymer ~30% | | | | | | |
| | aqueous solution | | | | | | |
| Demin. water | Water | to 100 | to 100 | to 100 | to 100 | to 100 | to 100 |
| Cetiol ® | Unciecan (and) | 1.00 | 1.00 | | | 0.50 | |
| Ultimate | triciecane | | | | | | |
| Cyclomethicone | Cyclomethicone | | | | | 0.50 | |
| Tocopherol | Tocopherol | 0.10 | | | 0.20 | | 0.50 |
| | (Vitamin E) | | | | | | |
| Cegesoft ® | Olus Oil | | 0.2 | | | 0.1 | |
| PS 6 | | | | | | | |
| Tinosorb ® M | Methylen Bis- | 1.00 | | | 1.00 | | |
| | Benzotriazolyl | | | | | | |
| | Tetramethylbutyl- | | | | | | |
| | phenol | | | | | | |
| | (and) Aqua | | | | | | |
| | (and) Decyl | | | | | | |
| | Glucoside (and) | | | | | | |
| | Propylene Glycol | | | | | | |
| | (and) Xanthan Gum | | | | | | |
| Z-Cote ® HP1 | Zinc Oxide (nano), | | 1.00 | | | 1.00 | |
| | Triethoxycaprylylsilane | | | | | | |
| T-80AS | Titanium Dioxide, | | | 1.00 | | | |
| | silica, triethoxy | | | | | | |
| | caprylylsilane | | | | | | |
| D-Panthenol | Panthenol | | 0.5 | | | | 0.5 |
| USP | | | | | | | |
| Glycerol | Glycerol | 3.00 | 5.00 | 1.00 | 2.0 | 1.00 | |
| PatcH2O ® | Water (and) glycerol | | | | | | |
| | (and) trehalose | | | | | | |
| | (and) urea (and) | | | | | | |
| | serine (and) | | | | | | |
| | pentylene glycol | | | | | | |
| | (and) glyceryl | | | | | | |
| | polyacrylate (and) | | | | | | |
| | algin (and) caprylyl | | | | | | |
| | glycol (and) sodium | | | | | | |
| | hyaluronate (and) | | | | | | |
| | pullulan (and) | | | | | | |
| | disodium phosphate | | | | | | |
| | (and) potassium | | | | | | |
| | phosphate | | | | | | |
| Hydagen ® GG | Glyceryl glucoside | | | | | 5.00 | 12.00 |
| | and glycerol | | | | | | |
| PURISOFT | Water (and) glycerol | | 3.00 | | 1.00 | | 1.50 |
| POE LS 9726 | (and) Moringa | | | | | | |
| | pteryosperma seed | | | | | | |
| | extract | | | | | | |
| Talc FM SSA | Talc (and) | 1.00 | | | 1.00 | | |
| (K.S. Pearl) | dimethicone | | | | | | |
| Mearlmica ® | Mica, lauroyl lysine | | 2.00 | | | | 1.00 |
| treated SVA | | | | | | | |
| Pearl-Glo ® SF | Bismuth oxychloride | | | 1.00 | | | |
| PG1099 | | | | | | | |
| SA-SB-300 | Silica | | | | 0.5 | | |
| (Miyoshi | | | | | | | |
| kasei) | | | | | | | |
| Dow Corning | Dimethicone/vinyl | | | | | 2.00 | 1.00 |
| 9701 | dimethicone | | | | | | |
| Cosmetic | crosspolymer (and) | | | | | | |
| powder (Dow | Silica | | | | | | |
| Corning) | | | | | | | |
| STR-100A-LP | Titanium dioxide | | | | | 5.00 | |
| (Sakai | (nano), hydrated | | | | | | |
| Chemical | silica, dimethicone/ | | | | | | |
| Industry) | methicone | | | | | | |
| | copolymer, | | | | | | |
| | aluminum hydroxide | | | | | | |
| Timica ® Terra | Mica, titanium | 1.00 | 0.5 | 1.00 | 0.5 | 0.5 | |
| White | dioxide | | | | | | |
| MN4501 | | | | | | | |
| Timica ® Terra | Mica, iron oxides, | 0.1 | 0.1 | | | | |
| Brown | titanium dioxide | | | | | | |
| MN4509 | | | | | | | |
| Timic ® Terra | Mica, iron oxides, | | 0.1 | 0.1 | | 0.1 | |
| Yellow | titanium dioxide | | | | | | |
| MN4502 | | | | | | | |
| Flamenco ® | Mica, titanium | | | | | 0.5 | 1.00 |
| Silk Blue | dioxide | | | | | | |
| 630M | | | | | | | |
| KOH 20% | Potassium | to adjust to pH 6.5-7.0 | | | | | |
| | hydroxide | | | | | | |
| Euxyl ® K 320 | 2-Phenoxyethanol, | | | 1.00 | 1.30 | 1.30 | 1.00 |
| | methyl 4- | | | | | | |
| | hydroxybenzoate, | | | | | | |
| | ethyl 4- | | | | | | |
| | hydroxybenzoate | | | | | | |
| Protectol ® PE | Phenoxyethanol | 1.00 | 1.00 | | | | |
| Sensive ® | Ethylhexylglycerin | 0.50 | 0.50 | | | | |
| SC50 | | | | | | | |
| pH | | 6.8 | 6.6 | 6.5 | 6.4 | 6.5 | 6.6 |

### Formulations 14-19: Creams

As skin protection agent for day care, baby care, facial care and body care

| | Chemical name/ | | | | | | |
|---|---|---|---|---|---|---|---|
| Constituents | compostion (%) | 14 | 15 | 16 | 17 | 18 | 19 |
| Polymer 1 | | 0.2 | 0.2 | 0.1 | 0.5 | 0.1 | 0.5 |
| Tinovis ® | Hydrophobically | | 3.33 (= 1 | 1.65 (= 0.1 | 1.65 (= 0.1 | 0.33 (= 0.1 | 0.33 (= 0.1 |
| GTC UP | modified copolymers | | active | active | active | active | active |
| | of acrylic acid | | content) | content) | content) | content) | content) |
| | Acrylate/Beheneth-25 | | | | | | |
| | Methacrylate | | | | | | |
| | Copolymer ~30% | | | | | | |
| | aqueous solution | | | | | | |
| Demin. water | Water | to 100 | to 100 | to 100 | to 100 | to 100 | to 100 |
| Emulgade PL | Cetearyl Glucoside and | 1.00 | | 1.00 | | | |
| 68/50 | Cetearyl Alcohol | | | | | | |
| Emulgade | Sucrose Polystearate | | 1.50 | | | | |
| Sucro Plus | and Cetyl Palmitate | | | | | | |
| Eumulgin VL | Lauryl Glucoside (and) | | | | 1.50 | | |
| 75 | Polyglyceryl-2 | | | | | | |
| | Dipolyhydroxystearate | | | | | | |
| | (and) Glycerol | | | | | | |
| Eumulgin | Disodium Cetearyl | | | | | 0.2 | |
| Prisma | Sulfosuccinate | | | | | | |
| AXOL ® C 62, | Glyceryl Stearate | | | | | | 1.00 |
| Evonik | Citrate | | | | | | |
| Cetiol ® | Unciecan (and) | 2.50 | 2.50 | 1.25 | 5.00 | 0.50 | 2.50 |
| Ultimate | triciecane | | | | | | |
| Cyclomethicone | Cyclomethicone | | | | | 2.50 | 0.50 |
| Tocopherol | Tocopherol | 0.10 | | | 0.20 | | 0.50 |
| | (Vitamin E) | | | | | | |
| Cegesoft ® | Olus Oil | | 0.20 | | | 0.1 | |
| PS 6 | | | | | | | |
| Tinosorb ® M | Methylen Bis- | 1.00 | | | 5.00 | | |
| | Benzotriazolyl | | | | | | |
| | Tetramethylbutyl- | | | | | | |
| | phenol | | | | | | |
| | (and) Aqua | | | | | | |
| | (and) Decyl | | | | | | |
| | Glucoside (and) | | | | | | |
| | Propylene Glycol | | | | | | |
| | (and) Xanthan Gum | | | | | | |
| Z-Cote ® HP1 | Zinc Oxide (nano), | | 1.00 | | | 1.00 | |
| | Triethoxycaprylylsilane | | | | | | |
| T-80AS | Titanium Dioxide, | | | 1.00 | | | |
| | silica, triethoxy | | | | | | |
| | caprylylsilane | | | | | | |
| D-Panthenol | Panthenol | | 0.5 | | | | 0.5 |
| USP | | | | | | | |
| Glycerol | Glycerol | 3.00 | 5.00 | 1.00 | 2.00 | 1.00 | |
| PatcH2O ® | Water (and) glycerol | | | | 1.50 | | 3.00 |
| | (and) trehalose | | | | | | |
| | (and) urea (and) | | | | | | |
| | serine (and) | | | | | | |
| | pentylene glycol | | | | | | |
| | (and) glyceryl | | | | | | |
| | polyacrylate (and) | | | | | | |
| | algin (and) caprylyl | | | | | | |
| | glycol (and) sodium | | | | | | |
| | hyaluronate (and) | | | | | | |
| | pullulan (and) | | | | | | |
| | disodium phosphate | | | | | | |
| | (and) potassium | | | | | | |
| | phosphate | | | | | | |
| Hydagen ® GG | Glyceryl glucoside | | | | | 5.00 | 5.00 |
| | and glycerol | | | | | | |
| PURISOFT | Water (and) glycerol | | 3.00 | | 1.00 | | 1.50 |
| POE LS 9726 | (and) Moringa | | | | | | |
| | pteryosperma seed | | | | | | |
| | extract | | | | | | |
| Talc FM SSA | Talc (and) | 1.00 | | | 1.00 | | |
| (K.S. Pearl) | dimethicone | | | | | | |
| Mearlmica ® | Mica, lauroyl lysine | | 2.00 | | | | 1.00 |
| treated SVA | | | | | | | |
| Pearl-Glo ® SF | Bismuth oxychloride | | | 1.00 | | | |
| PG1099 | | | | | | | |
| KOH 20% | Potassium | to adjust | | | | | |
| | hydroxide | to pH 6.5-7.0 | | | | | |
| Euxyl ® K 320 | 2-Phenoxyethanol, | | | 1.30 | 1.30 | 1.00 | 1.00 |
| | methyl 4- | | | | | | |
| | hydroxybenzoate, | | | | | | |
| | ethyl 4- | | | | | | |
| | hydroxybenzoate | | | | | | |
| Protectol ® PE | Phenoxyethanol | 1.00 | 1.00 | | | | |
| Sensive ® | Ethylhexylglycerin | 0.50 | 0.50 | | | | |
| SC50 | | | | | | | |
| pH | 6.8 | 6.6 | 6.5 | 6.4 | 6.5 | 6.6 | |

1. A method for bringing about an anti-pollution effect on human skin or human hair comprising applying a cosmetic product to the human skin or human hair which requires an anti-pollution effect, wherein the cosmetic product comprises a polymer and additionally other known cosmetic ingredients,
wherein the polymer has a solubility in water of at least 0.01 g of polymer in 100 g of water at 20° C. at least at a pH in the range between 4 and 9,
and wherein the polymer is selected from the group consisting of
a copolymer of methacrylic acid and ethyl acrylate, which optionally also comprises repeating units which have been derived from at least one monomer selected from the group consisting of acrylic acid, methacrylamide, an alkyl acrylate having 2 to 30 carbon atoms in the alkyl group, and an alkyl methacrylate having 2 to 30 carbon atoms in the alkyl group, an ester of methacrylic acid with a C12- to C30 fatty alcohol, wherein the fatty alcohol has been ethoxylated with 1 to 100 ethylene oxide units, and a monomer comprising at least two polymerizable ethylenically unsaturated double bonds, to effect branching or crosslinking, and
a copolymer of vinylpyrrolidone with at least one further ethylenically unsaturated polymerizable monomer, wherein a proportion of vinylpyrrolidone in said copolymer is at most 90% by weight,
wherein in the case that the polymer comprises repeating units derived from methacrylic acid, said repeating units are present as free acids or as salts.

2. The method according to claim 1, wherein the monomer comprising at least two polymerizable ethylenically unsaturated double bonds is pentaerythritol triallyl ether.

3. The method according to claim 1, wherein the polymer is a copolymer of vinylpyrrolidone with at least one further ethylenically unsaturated polymerizable monomer, wherein the proportion of vinylpyrrolidone in said copolymer is at most 90% by weight, and wherein the at least one further ethylenically unsaturated polymerizable monomer is selected from the group consisting of vinylimidazole, methylvinylimidazolium together with any anion, acrylamide, and methacrylamide.

4. The method according to claim 3, wherein the anion is selected from the group consisting of chloride and (H₃C—O—SO₃)⁻.

5. The method according to claim 1, wherein the polymer has a glass transition temperature of at least 70° C.

6. The method according to claim 1, wherein the polymer has an average molar mass (weight average Mw) of at least 100,000 g/mol.

7. The method according to claim 1, wherein the polymer is selected from the group consisting of
a copolymer composed of 30-60% by weight ethyl acrylate, 30-60% by weight methacrylic acid, 5-30% by weight methacrylamide, 0.1-10% by weight C18-alkyl-(EO)25 methacrylates and 0.01-1.0% by weight pentaerythritol triallyl ether,
a copolymer composed of 30-60% by weight ethyl acrylate and 40-70% by weight methacrylic acid,
a copolymer composed of 40-60% by weight ethyl acrylate, 30-50% by weight methacrylic acid, 5-15% by weight acrylic acid, and 0.01-1.0% by weight pentaerythritol triallyl ether,
a terpolymer composed of 60-80% by weight tert-butyl acrylate, 5-20% by weight ethyl acrylate, and 10-30% by weight methacrylic acid,
a copolymer composed of 40-65% by weight vinylpyrrolidone, 25-35% by weight methacrylamide, 5-20% by weight vinylimidazole, and 5-20% by weight quaternary (methylated) vinylimidazole, and
a copolymer composed of 50-70% by weight vinylpyrrolidone, 20-40% by weight methacrylamide and 1-10% by weight vinylimidazole.

8. The method according to claim 1, wherein the polymer is present in the cosmetic product at a concentration of 0.01 to 5% by weight.

9. The method according to claim 8, wherein the cosmetic product comprises at least water as the additionally other known cosmetic ingredient in an amount of at least 20% by weight, and wherein the polymer is present completely dispersed, suspended, dissolved, or swollen in water at 20° C.

10. The method according to claim 1, wherein the cosmetic product serves to protect, to care for, and/or to cleanse skin or to care for and/or to cleanse hair.

11. The method according to claim 1, wherein the cosmetic product is selected from the group consisting of an aqueous polymer solution, an emulsifier-free emulsion, and an emulsion.

12. The method according to claim 1, wherein the anti-pollution effect is protection from dust.

13. The method of claim 13 wherein the dust is a fine dust.
